(19) United States
(12) Patent Application Publication
(10) Pub. No.: US 2025/0195882 A1
(43) Pub. Date: Jun. 19, 2025

(54) MUSCLE OR NERVE FUNCTION REHABILITATION ASSISTANCE DEVICE USING CONDUCTIVE HYDROGEL

(71) Applicant: Research & Business Foundation Sungkyunkwan University, Suwon-si (KR)

(72) Inventors: Mikyung SHIN, Suwon-si (KR); Donghee SON, Suwon-si (KR); Subin JIN, Suwon-si (KR); Heewon CHOI, Suwon-si (KR)

(73) Assignee: Research & Business Foundation Sungkyunkwan University, Suwon-si (KR)

(21) Appl. No.: 18/980,747

(22) Filed: Dec. 13, 2024

(30) Foreign Application Priority Data
Dec. 15, 2023 (KR) 10-2023-0183594

(51) Int. Cl. A61N 1/36; A61L 27/44; A61L 27/52

(52) U.S. Cl. CPC A61N 1/36003; A61L 27/446; A61L 27/52; A61N 1/36139; A61N 1/3615; A61L 2400/06; A61L 2400/12; A61L 2430/30; A61L 2430/32

(57) ABSTRACT

The present disclosure relates to a muscle or nerve function rehabilitation assistance device using electrically conductive hydrogel. The electrically conductive hydrogel (IT-IC) provides an electrophysiological interface function between electrically active tissues, enabling the filling of lost muscle or nerve tissue for immediate rehabilitation in the early stages. It facilitates electrophysiological signal transmission between tissues and accelerates myofiber regeneration during the recovery phase. Particularly, when the electrically conductive hydrogel (IT-IC) is applied, the transmission of electrical signals generated in the muscle can be precisely controlled by a closed-loop device capable of being feedback in conjunction with peripheral nerve stimulation and electromyogram (EMG) monitoring.

### CROSS-REFERENCE TO RELATED APPLICATION(S)

This application is based on and claims priority under 35 U.S.C. 119 to Korean Patent Application No. 10-2023-0183594, filed on Dec. 15, 2023, in the Korean Intellectual Property Office, the disclosure of which is herein incorporated by reference in its entirety.

### BACKGROUND OF THE INVENTION

### 1. Field of the Invention

This invention was conducted with the support of the Ministry of Science and ICT of the Republic of Korea under project number 00215627. The research management agency for the above project is the Korea Regenerative Medicine Technology Development Project Group. The project name is “Regenerative Medicine Technology Development Project”, and the research task title is “Development of Injectable, Nerve-Adhesive, Biodegradable Hydrogel Electrodes for Wireless Electrical Stimulation to Promote Peripheral Nerve Damage Regeneration in a Minimally Invasive Manner”. The principal institution is Sungkyunkwan University Research & Business Foundation, and the research period is from Apr. 1, 2023, to Dec. 31, 2023.

The present disclosure relates to a muscle or nerve function rehabilitation assistance device using conductive hydrogel.

### 2. Description of the Prior Art

In cases of muscle injury, if proper treatment is not provided in the early stages, skeletal muscle may be replaced by non-contractile fibrous tissue, leading to chronic loss and impairment of muscle function. Therefore, appropriate rehabilitation therapy in the early stages is critical for the long-term normal recovery of muscles. However, restoring the movement and functionality of damaged muscles in the early stages of injury remains a challenging task.

In this context, the development of lightweight exoskeletons and wearable or implantable devices integrated with closed-loop gait rehabilitation technology plays a significant role in the effective recovery from motor and sensory dysfunctions associated with severe nerve and muscle injury. However, most bioelectronic devices developed for neural and muscular tissue interfaces to date are based on patch-type formfactors, posing significant limitations in applying them to narrow, complex, and small biological interfaces at the site of injury.

Conductive hydrogels that can be injected using syringes have been studied for years in tissue engineering and soft bioelectronics because they can be effectively introduced into inaccessible areas. However, most injectable conductive materials, consisting of reversible bonds with embedded electrically conductive additives, exhibit poor mechanical/electrical durability due to weak intermolecular bonding and uneven distribution of conductive substances, hindering stable on-site tissue interfacing in the long term. To address this issue, strong irreversible covalent bonds have been introduced to improve the mechanical properties of existing hydrogels. However, this approach has reduced the injectability of hydrogels.

Accordingly, there is a pressing need to develop a new bioelectronic device platform that can easily be applied to while the site of injury assisting in immediate rehabilitation by replacing the functions of lost tissue and enabling smooth interactions with the remaining tissues.

### RELATED ART DOCUMENT

### Patent Literature

U.S. Pat. No. 9,770,337B2 (issued Sep. 26, 2017)
Korean Patent No. 10-2022-0013894 A (issued Feb. 4, 2022)

### SUMMARY OF THE INVENTION

Leading to the present disclosure, intensive and thorough research conducted by the present inventors with the aim of developing a gait rehabilitation assistance device for the efficient recovery of motor and sensory functions associated with nerve damage and muscle injury, resulted in designing a highly biocompatible, reversibly conductive hyaluronic acid-based hybrid hydrogel that matches the mechanical modulus and electrophysiological properties of biological tissues and which was identified to have the effectiveness of restoring movement in the early stages of injury and achieving long-term recovery of motor and sensory functions a robot-assisted using closed-loop gait rehabilitation device.

Accordingly, the present disclosure aims to provide a device that assists in the effective rehabilitation of muscles and nerves using conductive hydrogel.

Ultimately, the present disclosure is to enable the efficient recovery of motor and sensory functions associated with nerve damage and muscle injury through the above-mentioned gait rehabilitation assistance device.

The present inventors have devoted extensive research efforts to developing a muscle or nerve function rehabilitation assistance device for the efficient recovery of motor and sensory functions associated with nerve damage and muscle injury. As a result, a highly biocompatible, reversibly conductive hyaluronic acid-based hybrid hydrogel was designed to match the mechanical modulus and electrophysiological properties of biological tissues and identified to have the effectiveness of restoring movement in the early stages of injury and achieving long-term recovery of motor and sensory functions using a robot-assisted closed-loop rehabilitation device.

According to one aspect thereof, the present disclosure provides a muscle or nerve function rehabilitation assistance device 1 comprising:
(a) a conductive hydrogel to be injected into a muscle injury site or nerve injury site of a subject;
(b) a neural stimulation electrode 100 for delivering electrical stimulation to the subject's nerves;
(c) an electrical stimulation generator 200 for applying a stimulation signal to the neural stimulation electrode;
(d) an electromyographic electrode 300 for measuring an electromyographic signal of the target rehabilitation muscle of the subject; and
(e) an electromyographic recorder 400 for receiving an electromyographic signal from the electromyographic electrode.

In an embodiment of the present disclosure, the neural stimulation electrode is directly attached to the nerve of the subject or comes into contact with the conductive hydrogel injected into the nerve injury site.

In an embodiment of the present disclosure, the electromyographic electrode is directly attached to the target rehabilitation muscle of the subject or comes into contact with the conductive hydrogel injected into the muscle injury site.

In yet another embodiment of the present disclosure, the muscle or nerve function rehabilitation assistance device further includes: (f) a motor function assistance device 500 for rehabilitating the motor functionality of the muscle.

The motor function assistance device 500 includes: an actuator 503 for providing movement assistance force in the flexion-extension direction of the muscle of the subject; a body part fixing unit 504 for securing a body part of the subject; a connecting member 505 for transmitting force by linking the actuator and the body part fixing unit; and a power control unit 506 for determining whether to supply power to the actuator.

The motor function assistance device may serve as a gait assistance device.

When functioning as a gait assistance device, the motor function assistance device may further include a treadmill 501 for the gait of the subject and a support (502) capable of supporting the weight of the subject.

That is, the gait assistance device includes a treadmill 501 in which the gait of the subject is carried out, a support 502 for supporting the weight of the subject, an actuator 503 for providing gait assistance force to a leg of the subject, a leg fixing unit 504 for securing the leg of the subject, and a connecting member 505 for transmitting force by linking the actuator and the leg fixing unit.

In an embodiment of the present disclosure, the subject is a rehabilitation target with damage to the muscles or nerves of the lower extremities.

In a specific embodiment of the present disclosure, the subject has lost sensory and motor functions due to damage to the muscles or nerves of the lower extremities and requires gait rehabilitation.

Additionally, in an embodiment of the present disclosure, the subject has conductive hydrogel injected into the muscle or nerve injury site.

In an embodiment of the present disclosure, the conductive hydrogel is characterized by including hyaluronic acid and noble metal nanoparticles, where the hyaluronic acid forms a network connected by biphenyls, and the noble metal nanoparticles are dispersed within the network.

Conductive hydrogels are generally promising materials for tissue engineering applications in the heart, muscles, and nerves, drug delivery devices responsive to electrical signals, and bioelectronics. To manufacture conductive hydrogels, methods have been devised to insert electrically conductive additives such as carbon nanotubes, graphene, noble metal nanoparticles, and conductive polymers into cross-linked hydrogel networks. However, these conductive additives exhibit several disadvantages, including poor solubility in hydrophilic environments, cytotoxicity, low dispersion within the hydrogel network, poor gelation, irreversible conductivity, and weak mechanical properties. To overcome these issues, prior patents of the inventors have developed electrically conductive hydrogels using biocompatible natural polymers and provided methods for their production.

Ultimately, the present disclosure provides an application method for using an electrically conductive hydrogel (IT-IC) as an injectable tissue interface implant for muscle or nerve function rehabilitation assistance devices.

In an embodiment of the present disclosure, the IT-IC hydrogel is applied to damaged muscle and nerve tissue regions to provide electrophysiological interface functionality between electrically active tissues, thus allowing for filling the loss of damaged muscle tissue in the early stages for immediate rehabilitation, enabling electrophysiological signal transmission between tissues, and accelerating myofiber regeneration during the recovery phase.

Additionally, in an embodiment of the present disclosure, the muscle or nerve function rehabilitation assistance device using the IT-IC hydrogel may be provided as a robot-assisted rehabilitation device that precisely controls the delivery of electrical signals generated in the subject's damaged muscles through a closed-loop system capable of peripheral nerve stimulation and electromyography (EMG) monitoring.

In an embodiment of the present disclosure, the conductive hydrogel includes hyaluronic acid polymer as a backbone, with phenylboronic acid grafted thereto.

In an embodiment of the present disclosure, the degree of grafting between the hyaluronic acid polymer and phenylboronic acid may range from 2% to 15%, but is not limited thereto.

The IT-IC hydrogel used in the present disclosure is prepared by mixing a solvent, a hyaluronic acid polymer, and a boronic acid precursor containing phenyl groups to create an HA-BA polymer solution including hyaluronic acid polymer as a backbone and phenylboronic acid as a graft. Subsequently, an alkaline solution and noble metal ions are added to the HA-BA polymer solution to include reduced noble metal nanoparticles.

In this regard, the solvent may include water, ethanol, or a combination thereof, but is not limited thereto.

In addition, the hyaluronic acid polymer used herein represents a biocompatible natural polymer, which is found as a constituent in the vitreous, skin, extracellular matrix, etc.

In substitution for the hyaluronic acid polymer, any selected from the group consisting of cellulose, collagen, chitin, chitosan, keratin, silk, elastin, and combinations thereof may be used as an alternative, provided they exhibit biocompatibility and conductive polymeric properties.

In an embodiment of the present disclosure, the HA-BA polymer solution may further include 1-ethyl-3-(3-dimethylaminopropyl)-carbodiimide hydrochloride (EDC) and N-hydroxysuccinimide (NHS), but with no limitations thereto.

EDC and NHS facilitate the removal of hydrogen from carboxyl groups, enabling bonding with amine groups. Specifically, when EDC removes hydrogen from the carboxyl group of hyaluronic acid, EDC binds to the hyaluronic acid at the position of the removed hydrogen. Subsequently, the OH group of NHS binds to the EDC position, and phenylboronic acid containing an amine group bonds with the hyaluronic acid.

For example, the boronic acid precursor containing phenyl groups may include aminophenylboronic acid (C₆H₄NH₂B(OH)₂), but is not limited thereto.

In an embodiment of the present disclosure, the noble metal ions may include ions selected from the group consisting of Au, Ag, Pt, Pd, Ir, Os, or combinations thereof. Preferably, the noble metal ion is Au³⁺, and the precursor of Au³⁺ ions may be HAuCla, but with no limitations thereto.

In an embodiment of the present disclosure, the alkaline solution may include one selected from the group consisting of NaOH, KOH, Ba(OH)₂, Ca(OH)₂, NH₄OH, and combinations thereof. Preferably, the alkaline solution includes NaOH, but is not limited thereto.

The molar ratio of OH— in the alkaline solution to the noble metal ions affects the formation of the biphenyl structure during the manufacture of the electrically conductive hydrogel of the present disclosure. If the molar ratio is too low, for example, less than 0.1, the deboronylation reaction is insufficient, resulting in fewer biphenyl structures and a hydrogel with low storage modulus (G′). Conversely, if the molar ratio is too high, for example, greater than 10, the abundant OH-restrains the deboronylated polymer from reducing the noble metal ions to noble metal nanoparticles, but allows for the progression of noble metal ions to noble nanoparticles. In greater detail, when reduced by the deboronylated polymer, the noble ions can form a biphenyl structure, but when the noble ions are reduced by OH—, the deboronylated polymer can be restrained from forming a biphenyl structure.

Accordingly, the molar ratio of OH— to noble metal ions in the alkaline solution may range from 0.1 to 10, but is not limited thereto. For example, the molar ratio of OH— to noble metal ions in the alkaline solution may be about 0.1 to about 10, about 1 to about 10, about 2 to about 10, about 3 to about 10, about 4 to about 10, about 5 to about 10, about 6 to about 10, about 7 to about 10, about 8 to about 10, about 9 to about 10, about 0.1 to about 1, about 0.1 to about 2, about 0.1 to about 3, about 0.1 to about 4, about 0.1 to about 5, about 0.1 to about 6, about 0.1 to about 7, about 0.1 to about 8, about 0.1 to about 9, about 1 to about 9, about 2 to about 8, about 3 to about 7, about 4 to about 6, or about 5, but with no limitations thereto.

Additionally, the diameter and size distribution of noble metal nanoparticles, e.g., AuNP, formed in the hydrogel of the present disclosure may vary depending on the molar ratio of the alkaline solution to the noble metal ions (e.g., NaOH/Au³⁺ ratio).

Addition of the alkaline solution and the precursor of noble metal ions to the HA-BA polymer solution induces temporary shear-thinning characteristics according to the stoichiometric ratio with the noble metal ion precursor, leading to enhanced viscosity in the resulting conductive hydrogel through cross-linking within the HA-BA polymer.

In an embodiment of the present disclosure, EDC/NHS is added to a solution of hyaluronic acid polymer in a solvent, followed by the addition of a boronic acid precursor containing phenyl groups. Through the coupling reaction of EDC/NHS, the boronic acid precursor containing phenyl groups is conjugated and grafted to the side groups of the hyaluronic acid polymer to produce HA-BA polymers. Subsequently, noble metal ions (e.g., HAuCl₄) and an alkaline solution (e.g., NaOH) are added to the HA-BA polymer solution to produce an electrically conductive hydrogel (IT-IC).

FIG. 2 is a schematic diagram illustrating a method for manufacturing the electrically conductive hydrogel (IT-IC) used in the present disclosure.

In an embodiment of the present disclosure, the electrically conductive hydrogel is characterized by being easily injectable. Specifically, when injected between the ends of severed muscles or onto the rough surface or three-dimensional (3D) curves of damaged muscles, the hydrogel demonstrates excellent conformal contact, making it advantageous as a stable interface material for biological and non-biological substrates.

In an embodiment of the present disclosure, during the hydrogel formation process, HA-BA polymers are crosslinked to form the hydrogel and noble metal ions are reduced to form noble metal nanoparticles.

With ionic conductivity imparted thereto, these noble metal nanoparticles enable the hydrogel to have electrical conductivity, and their biocompatibility resolves the toxicity issues associated with conventional conductive hydrogels.

Furthermore, the conductive hydrogel of the present disclosure comprises hyaluronic acid, providing biocompatibility and electrical conductivity, which align with the mechanical modulus and electrophysiological characteristics of biological tissues, so that the hydrogel can found applications in tissue engineering and related fields.

In an embodiment of the present disclosure, the electrical conductivity of the hydrogel increases as the average cross-sectional area of the hydrogel at the site of injection, perpendicular to the direction of nerve or muscle travel, increases. This increase in conductivity can be represented by a reduction in resistance.

Bulk hydrogels prepared in an example of the present disclosure were injected line by line using needles with different thicknesses (18 G, 23 G, and 26 G), and measured for electrical resistance. Lower resistance values were detected in hydrogels injected using thicker needles (18 G), and resistance decreased as the volume of injected hydrogel increased.

In an embodiment of the present disclosure, the resistance of the hydrogel may range from 1 kΩ to 100 kΩ.

Specifically, the resistance may be about 1 kΩ to about 100 kΩ, about 10 kΩ to about 100 kΩ, about 20 kΩ to about 100 kΩ, about 30 kΩ to about 100 kΩ, about 40 kΩ to about 100 kΩ, about 50 kΩ to about 100 kΩ, about 60 kΩ to about 100 kΩ, about 70 kΩ to about 100 kΩ, about 80 kΩ to about 100 kΩ, about 90 kΩ to about 100 kΩ, about 1 kΩ to about 10 kΩ, about 1 kΩ to about 20 kΩ, about 1 kΩ to about 30 kΩ, about 1 kΩ to about 40 kΩ, about 1 kΩ to about 50 kΩ, about 1 kΩ to about 60 kΩ, about 1 kΩ to about 70 kΩ, about 1 kΩ to about 80 kΩ, about 1 kΩ to about 90 kΩ, about 10 kΩ to about 90 kΩ, about 20 kΩ to about 80 kΩ, about 30 kΩ to about 70 kΩ, about 40 kΩ to about 60 kΩ, or about 10 kΩ to about 50 kΩ, preferably for about 10 kΩ to about 50 kΩ.

In another embodiment of the present disclosure, the tangent delta (tan(δ)) of the conductive hydrogel may be greater than 0 and less than or equal to 0.5, but is not limited thereto.

The term “tangent delta” (tan(δ)) refers to the ratio of the loss modulus of a material in a pure viscous liquid state to the storage modulus of the material in full elastomeric state, indicating the rate of energy loss during vibration.

Lower tangent delta means more material firmness. In general, gels with a tangent delta above 0.5 are classified as soft gels, while those below 0.5 are considered firm gels.

In an embodiment of the present disclosure, the elongation of the conductive hydrogel in the longitudinal direction may range from 80% to 120%, but with no limitations thereto.

The term “elongation” refers to the ratio of the maximum stretched length without breaking after stretching in the longitudinal direction, to the original length before stretching.

Stretching the hydrogel increases its length and decreases its cross-sectional area, potentially increasing resistance. Thus, the resistance of the conductive hydrogel may increase with its stretched length, but is not limited thereto.

In an embodiment of the present disclosure, the conductive hydrogel functions as a junction mediating physical movements, including muscle relaxation and contraction.

In an embodiment of the present disclosure, the electrical stimulation generator 200 delivers electrical stimulation through the neural stimulation electrode 100, inducing muscle contraction signals in muscles of the subject.

In an embodiment of the present disclosure, the neural stimulation may be transmitted via the conductive hydrogel injected into a nerve region of the subject.

In an embodiment of the present disclosure, the muscle contraction signals may be transmitted in the form of electromyogram (EMG) signals through the electromyographic electrode 300.

In an embodiment of the present disclosure, the EMG signals are transmitted via the conductive hydrogel injected into a muscle region of the subject.

In an embodiment of the present disclosure, the electrical stimulation generator 200 can deliver electrical stimulation through the neural stimulation electrode 100 to induce muscle contraction and neuromuscular response signals in muscles of the subject.

For example, the muscles of the subject may have lost motor function due to damage, such as severance. By injecting the conductive hydrogel of the present disclosure into the damaged muscle area and applying external electrical stimulation, muscle contraction and neuromuscular response signals can be induced in the leg muscles of the subject. These induced muscle contraction and neuromuscular response signals can trigger contraction of the muscles of the subject.

The EMG signals generated by muscle contraction and neuromuscular response are transmitted from the electromyographic electrode attached to the conductive hydrogel injected into the muscle or muscle area to an external electromyographic recorder 400.

In an embodiment of the present disclosure, the electromyographic recorder 400 monitors and records the EMG signals and transmits the signals to the electrical stimulation generator 200 and the power control unit 506 of the motor function assistance device.

The electromyographic recorder filters out and removes noise signals generated by the subject's movements from the collected EMG signals.

In an embodiment of the present disclosure, the electrical stimulation generator 200 compares the EMG signals received from the electromyographic recorder with a predetermined reference value to control the magnitude of the stimulation signals.

In a specific embodiment of the present disclosure, the electrical stimulation generator 200 compares the target EMG signal value determined through electrical simulation with the measured EMG signal value to control the intensity of the generated electrical stimulation signal. Specifically, i) if the EMG signal is below the target EMG signal value, the magnitude of the electrical stimulation signal applied to the neural stimulation electrode is increased; ii) if the EMG signal exceeds the target EMG signal value, the magnitude of the electrical stimulation signal applied to the neural stimulation electrode is decreased.

In an embodiment of the present disclosure, the power control unit 506 compares the EMG signals received from the electromyographic recorder with a predetermined reference value and delivers motion assistance signals to the motor function assistance device 500.

In a specific embodiment of the present disclosure, the power control unit 506 compares the target EMG signal value determined through electrical simulation with the measured EMG signal value to control the intensity of the motion assistance force provided. Specifically, i) if the EMG signal is below the target EMG signal value, motion assistance signals are sent to the motor function assistance device 500 to increase the magnitude of the motion assistance force; ii) if the EMG signal exceeds the target EMG signal value, motion assistance signals are sent to the motor function assistance device 500 to decrease the magnitude of the motion assistance force.

In an embodiment of the present disclosure, the motion assistance signal received from the power control unit of the motor function assistance device is transmitted to the actuator of the motion assistance device.

As described above, the feature of the rehabilitation assistance device of the present disclosure, which adjusts the magnitude of electrical stimulation or motion assistance force by comparing the target and measured EMG signal values, can be described as a “closed-loop control system.”

In an embodiment of the present disclosure, the actuator 503 is characterized as a linear actuator operating in the flexion-extension direction of the muscle.

Additionally, in an embodiment of the present disclosure, if the motor assistance device is a gait assistance device, the actuator 503 provides force to assist the gait of the subject.

By applying the hydrogel of the present disclosure to subjects who have lost motor function due to damaged leg muscles, the electrical conductivity of the damaged area is activated, and external assistive force is provided, enabling effective operation of the gait rehabilitation assistance device.

In an embodiment of the present disclosure, the treadmill 501 can adjust the rotational speed thereof to match the subject's walking speed.

By adjusting the walking speed based on the subject's level of damage or r recovery stage, stepwise gait rehabilitation assistance may be applied.

In an embodiment of the present disclosure, the neural stimulation electrode 100 may be a cuff electrode but is not limited thereto.

In a specific embodiment of the present disclosure, the neural stimulation electrode serves as an intermediary for delivering neural stimulation from an external electrical stimulation generator to the subject. The electrode may be mounted on the surface of the target rehabilitation nerve or within the conductive hydrogel injected into the target nerve.

In an embodiment of the present disclosure, the electromyographic electrode 300 may be a stretchable electrode but is not limited thereto.

In a specific embodiment of the present disclosure, the electromyographic electrode may be mounted on the surface of the target rehabilitation muscle or within the conductive hydrogel injected into the target muscle.

In an embodiment of the present disclosure, the neural stimulation electrode 100 and the electromyographic electrode 300 may be coated with conductive hydrogel after being implanted into the subject. Alternatively, conductive hydrogel may be applied to the damaged area before the neural stimulation electrode 100 and the electromyographic electrode 300 are inserted.

In an embodiment of the present disclosure, neural stimulation through the neural stimulation electrode 100 may be applied with a strength of 0.05 to 1 V, but with no limitations thereto. The strength may be appropriately adjusted depending on the subject's size, type, and target rehabilitation muscle condition.

In an embodiment of the present disclosure, neural stimulation through the neural stimulation electrode 100 may be applied using specific pulses (0.1 to 10 ms in duration) at least once every 3 seconds, but with no limitations thereto. The interval between stimulations can be adjusted based on factors such as the subject's size, type, rehabilitation target muscle condition, and walking speed.

For example, the stimulation frequency may increase to up to 5 times per second depending on the walking speed, but with no limitations thereto.

In an embodiment of the present disclosure, the support structure may include handles that the subject can hold.

In another embodiment of the present disclosure, the support structure may include a fixing component to secure the subject's hands or upper body.

In an embodiment of the present disclosure, the muscle or nerve function rehabilitation assistance device of the present disclosure may be effectively applied to muscles or nerves with defects caused by injury or deformity.

In an embodiment of the present disclosure, the conductive hydrogel of the present disclosure may function as an implant when injected into a defective area, preventing cavity formation and atrophy of surrounding tissues caused by tissue loss.

In an embodiment of the present disclosure, the conductive hydrogel of the present disclosure efficiently transmits physiological signals through the conductivity thereof, promoting the recovery (rehabilitation) of movement and sensory functions through neural stimulation.

Furthermore, in an embodiment of the present disclosure, the conductive hydrogel of the present disclosure promotes the regeneration of damaged muscle or nerve tissues.

Recent studies (e.g., Jin, Y. et al. and Zhou, L. et al.) have reported that implanting conductive scaffolds supports muscle differentiation through efficient electrical conductivity. When IT-IC hydrogel is used to fill muscle injury sites, it can promote myogenesis and myoblast proliferation through the effective charge storage capacity thereof, even in the absence of cells or growth factors.

In an example of the present disclosure, electrically conductive hydrogel (IT-IC) was injected into the damaged muscle area of a mouse with volumetric muscle loss (VML) injury (8 mm in diameter). Successful muscle tissue regeneration was observed within four weeks.

Accordingly, by injecting the electrically conductive hydrogel of the present disclosure into damaged muscle areas, immediate treatment of nerves and muscles is enabled. When the electrically conductive hydrogel is applied to muscle or nerve function rehabilitation assistance devices, the signals transmitted through the conductivity can be recognized by external actuators, which provide assistive force to support the subject's gait. This increases the rehabilitation effects on the muscles or nerves in the damaged area. Unlike conventional methods that begin rehabilitation only after the damaged area is almost healed, the present disclosure facilitates early recovery and rehabilitation.

### Effect of the Invention

The electrically conductive hydrogel (IT-IC) of the present disclosure provides an electrophysiological interface function between electrically active tissues. It fills the loss of anterior tibialis muscle tissue in mice during the early stages of rehabilitation, enables electrophysiological signal transmission between tissues, and accelerates myofiber regeneration during the recovery phase. Particularly, when the electrically conductive hydrogel (IT-IC) of the present disclosure is applied, the transmission of electrical signals generated in the muscle can be precisely controlled by a closed-loop device capable of being feedback in conjunction with peripheral nerve stimulation and electromyogram (EMG) monitoring. This device can be utilized as a gait rehabilitation assistance system.

### BRIEF DESCRIPTION OF THE DRAWINGS

The above and other aspects, features and advantages of the present disclosure will be more apparent from the following detailed description taken in conjunction with the accompanying drawings, in which:

FIG. 1 is a schematic diagram of a closed-loop gait rehabilitation assistance device having electrically conductive hydrogel (IT-IC) applied thereto;

FIG. 2 is a schematic diagram illustrating the preparation of the electrically conductive hydrogel (IT-IC hydrogel) used in the gait rehabilitation assistance device;

FIG. 3 shows images verifying the conformal contact capability of the IT-IC hydrogel encapsulating fluorescein isothiocyanate (FITC) on rhodamine-stained skeletal muscle tissue (purple);

FIG. 4 shows electrical resistance measurements of the conductive hydrogel injected in lines using needles of different thicknesses (18 G, 23 G, and 26 G);

FIG. 5a shows images illustrating a process of connecting muscles by layering IT-IC hydrogel filaments injected line by line between severed muscles;

FIG. 5b is a graph showing EMG changes measured according to the injected conductive hydrogel;

FIG. 5c is a plot showing EMG changes measured based on stimulation voltage in a normal mouse (uninjured) and a mouse with severed muscles filled with conductive hydrogel (treated);

FIG. 6 depicts the treatment of muscle damage using conductive hydrogel: (6a) sequential processes from the creation of a volumetric muscle loss (VML) model in the anterior tibialis muscle in a mouse to the treatment of same with conductive hydrogel (IT-IC) and the regeneration of the muscle tissue after four weeks; (6b) cross-sections of anterior tibialis muscle stained with hematoxylin and eosin (H&E) at 1, 2, and 4 weeks after hydrogel treatment. Asterisks indicate myofibers with central nuclei; (6c) aspect ratios of regenerated anterior tibialis muscle fibers four weeks after hydrogel treatment; (6d) quantitative analysis of myofibers with central nuclei shown in (6b) (black: damaged, blue: treated with gelatin methacrylate (Gel-MA), red: treated with conductive hydrogel (IT-IC)); (6e): quantitative analysis aspect of ratios of regenerated myofibers shown in (6c);

FIG. 7 is a schematic diagram of a closed-loop robot-assisted rehabilitation device (C-RAR) using conductive hydrogel (IT-IC): a cuff electrode and IT-IC are placed on the sciatic peripheral nerve of a rat, and an epimysial electrode is placed on the anterior tibialis muscle; and nerve stimulation on the treadmill assists leg movement through robot assistance, with muscle signals triggering robot motion; and

FIG. 8 shows images illustrating rehabilitation training of rat treated for anterior tibialis muscle injury using IT-IC hydrogel, nerve stimulation, and robot-assisted gait training, and EMG measured during the training: (a) EMG of a rat walking without sciatic nerve stimulation; (b) gait impairment in the rat described in (a); (c) EMG of a rat walking with nerve stimulation; (d) the gait of the rat described in (c).

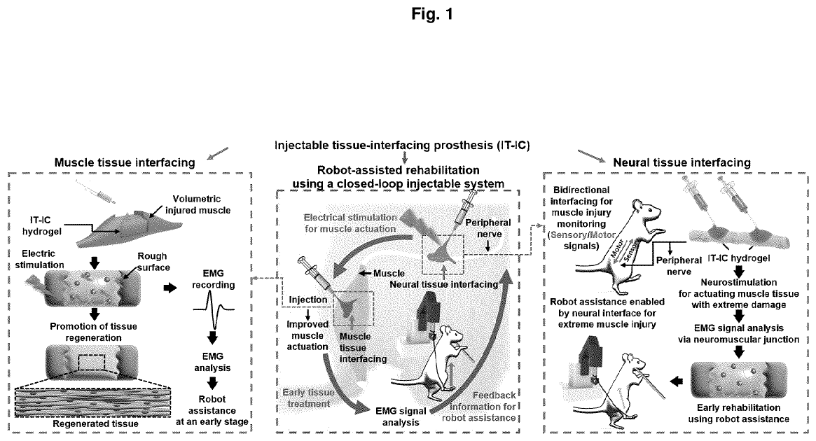

### DETAILED DESCRIPTION OF THE EXEMPLARY EMBODIMENTS

A better understanding of the present disclosure may be obtained through the following examples that are set forth to illustrate, but are not to construed to limit, the present disclosure.

### EXAMPLES

Throughout the specification, unless otherwise stated, the “%” used to represent the concentration of a specific substance refers to (weight/weight) % for solid/solid, (weight/volume) % for solid/liquid, and (volume/volume) % for liquid/liquid.

### Preparation Example 1: Preparation of Electrically Conductive Hydrogel (IT-IC)

To produce a soft conductive hydrogel resembling a biological tissue, hyaluronic acid was first dissolved in a solvent, and 4-aminophenyl boronic acid was conjugated to hyaluronic acid through an EDC/NHS coupling reaction to prepare HA-BA polymers. Then, NaOH and HAuCl₄ were added to the HA-BA polymer and stirred to produce a conductive hydrogel (IT-IC hydrogel) containing gold nanoparticles.

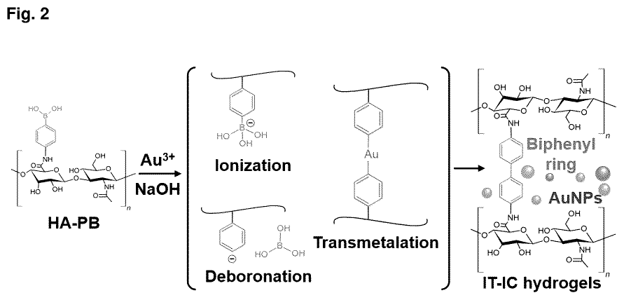

Briefly, hyaluronic acid (200 kDa) was dissolved in deionized water (DIW) to form a concentration of 10 mg/ml, and the pH was adjusted to 5.5. Then, 100 mg of EDC (equivalent to 95.85 mg relative to hyaluronic acid) was added to the solution and reacted for 20 minutes. Next, 100 mg of NHS (equivalent to 55.75 mg relative to hyaluronic acid) was added and reacted for 10 minutes. Subsequently, 100 mg of 4-aminophenylboronic acid (4-APB; equivalent to 86.7 mg relative to hyaluronic acid) was added to the solution and reacted for 24 hours. The resulting solution was placed inside a membrane with a molecular weight cutoff of 6 kDa to 8 kDa and dialyzed for 3 days using DIW containing NaCl at a concentration of 9 g/ml. The solution was then frozen at −8° C. for one day and subsequently freeze-dried at −80° C. for three days to produce the HA-BA solution. The HA-BA solution was then mixed with NaOH solution and HAuCl; while maintaining the pH around 8 and stirring to induce the reaction, resulting in the formation of the conductive hydrogel (IT-IC hydrogel) (FIG. 2).

### Example 1. Effect of Electrically Conductive Hydrogel (IT-IC)

The IT-IC hydrogel obtained in Preparation Example 1 was applied through injection between the ends of severed muscles or into damaged muscles.

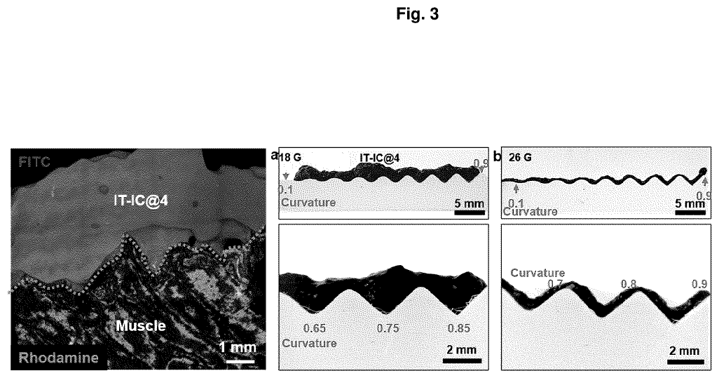

With ability to be adhesively injected onto narrow and rough surfaces, the IT-IC hydrogel of the present disclosure, when applied to such as the uneven surfaces of damaged biological tissues (e.g., muscles) or three-dimensional (3D) curved objects, demonstrated excellent conformal contact (FIG. 3).

### Example 1.1: Measurement of Electrical Resistance (Ω)

To evaluate the electrical resistance of the IT-IC hydrogel obtained in Preparation Example 1, bulk IT-IC hydrogel filament-like samples using needles with different gauges (diameter and length: 0.63 mm, 20 mm (18 G); 2.29 mm, 20 mm (23 G); 3.53 mm, 20 mm (26 G)). Electrical resistance (Ω) was measured using a four-point probe method (Keithley 2450 Source Meter) with a fixed probe spacing of 5 mm.

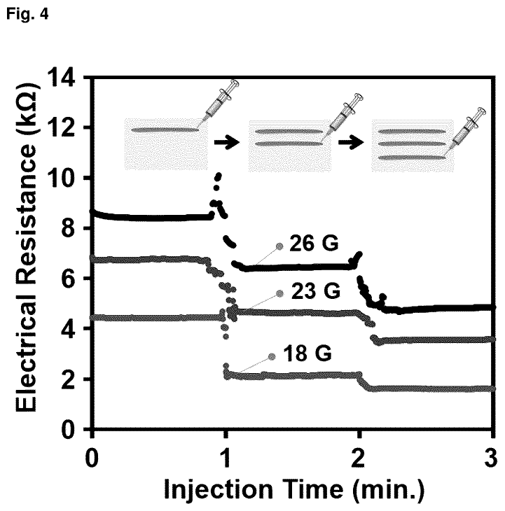

The results are shown in FIG. 4. As illustrated in FIG. 3, it was confirmed that the electrical resistance of the hydrogel injected through needles of all gauges (18 G, 23 G, and 26 G) decreased stably with each additional injection of hydrogel at 1-minute intervals.

Accordingly, it was demonstrated that electrical flow is continuously achieved through the IT-IC hydrogel, exhibiting excellent electrical conductivity.

### Example 1.2: Measurement of Electromyogram (EMG)

The charge transfer capability (EMG) of IT-IC hydrogel between muscle tissues was evaluated using an in vivo muscle defect model.

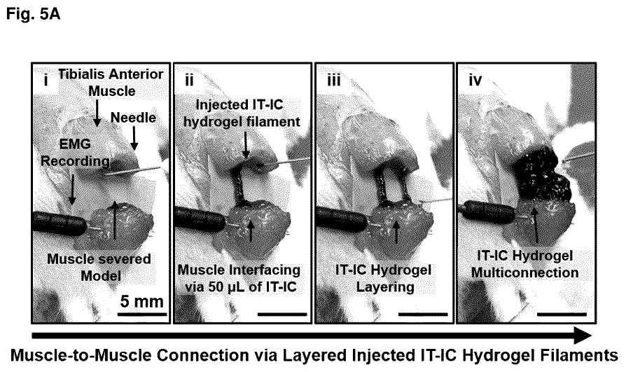

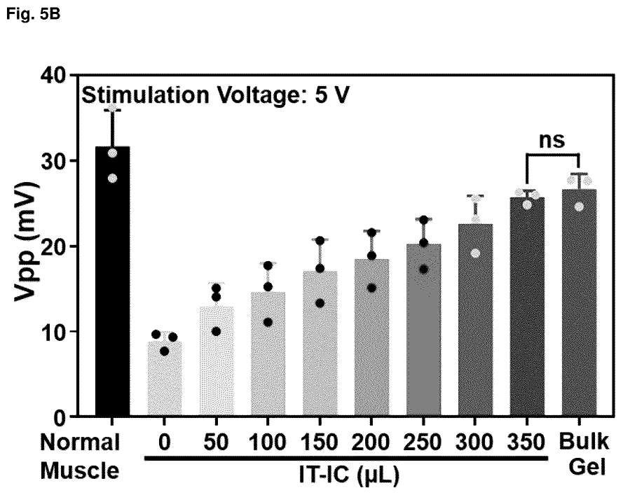

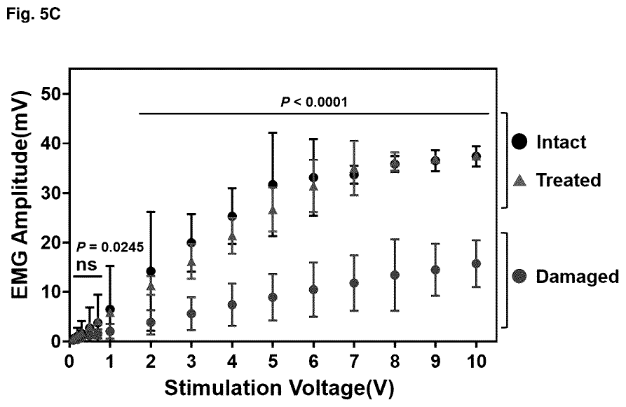

In brief, the anterior tibialis muscle of a mouse (SD mouse, male, 300-350 g, 10 weeks old) was isolated using Teflon tape to prevent electrical conduction to other muscles. The muscle was then severed using a scalpel, with the severed muscles separated by approximately 5 mm. The upper muscle received electrical stimulation from two pin-shaped single electrodes connected to a waveform generator. EMG signals transmitted to the lower muscle were recorded using a biosignal amplifier and data acquisition device while injecting IT-IC hydrogel into the defect area in real-time (total volume=400 μL, injected in 50 μL increments using a 26 G needle) (FIG. 5a). Two electrodes (cathode and anode) were inserted 2 cm apart into the lower anterior tibialis muscle, and a reference electrode was inserted into the tail.

The results are depicted in FIG. 5b.

As shown in FIG. 5b, when electrical stimulation was applied to the upper muscle of the severed muscle using a 26 G needle to inject 50 μL of IT-IC hydrogel filaments, the immediate recovery of electrophysiological function (EMG amplitude) was observed. Additionally, the amplitude increased with the amount of IT-IC hydrogel filaments injected. A total of 350 μL (bulk or layered filaments) of IT-IC hydrogel injected to a damaged muscle tissue exhibited a similar level of EMG amplitude (25.7±0.6 mV) to that of normal muscle tissue (31.7±3.4 mV).

From the EMG signal measurements between the severed muscles, it was confirmed that the viscous IT-IC hydrogel in a thin filament form factor could contact damaged interfaces and stably restore electrical flow between tissues.

To evaluate EMG recording capabilities of the IT-IC hydrogel, stimulation, recoding, and signal processing experiments were conducted on normal animals (SD rat, male, 300-350 g, 10 weeks old) as a positive control group, and rat without hydrogel (damaged) as a negative control group. In brief, IT-IC hydrogel was applied to the TA muscle of rat, and two pin electrodes for EMG recording were inserted. EMG signals were recorded while incrementally increasing the stimulation voltage.

The results are shown in FIG. 5c. When electrical stimulation was applied at voltages ranging from 0.1 V to 10 V, the EMG values measured in rat with IT-IC hydrogel injected between severed muscles were similar to those in normal rat. In contrast, the control group (damaged) without IT-IC hydrogel showed significant differences compared to the other groups (positive control and experimental group).

Thus, the electrically conductive hydrogel (IT-IC) was confirmed to effectively restore charge transfer capability between severed muscles to a normal level.

### Example 1.3: Evaluation of Muscle Regeneration Promotion

The muscle tissue repair (muscle regeneration promotion) effect of IT-IC hydrogel implantation was evaluated using a volumetric muscle loss (VML) model with a cylindrical defect (3 mm depth, 8 mm diameter) in the anterior tibialis muscle of SD rat (male, 300-350 g, 10 weeks old). Briefly, IT-IC or Gel-MA hydrogel (200 μL) was implanted into the defect area. The mice were sacrificed at 1, 2, and 4 weeks post-implantation, and the tissues were fixed in 4% (v/v) paraformaldehyde solution for histological analysis. Tissue samples were sectioned transversely or longitudinally, stained with hematoxylin and eosin (H&E), and the number of myofibers with central nuclei and the aspect ratio of myofibers were measured at randomly selected locations in the H&E-stained images.

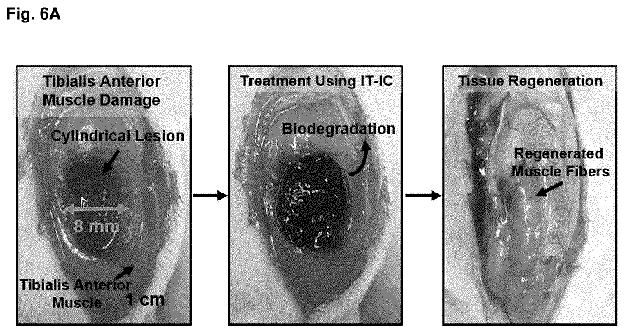

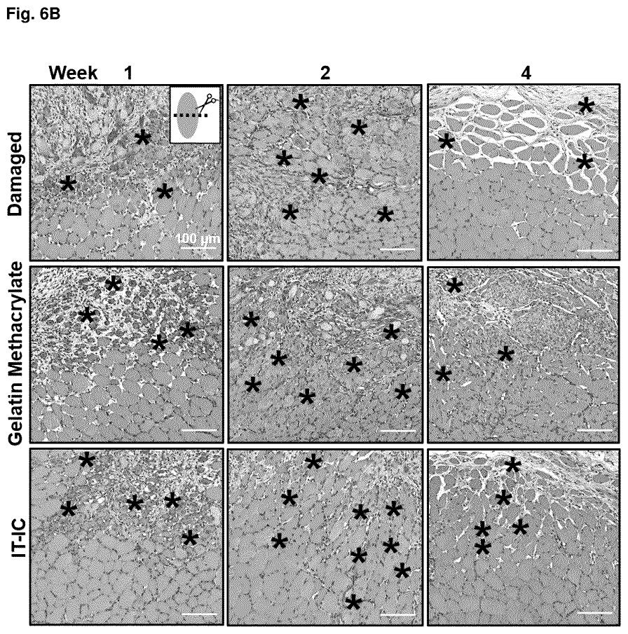

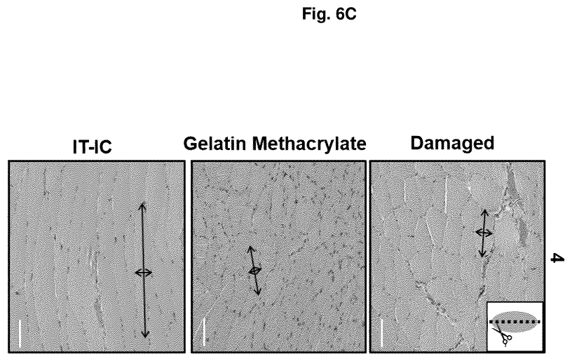

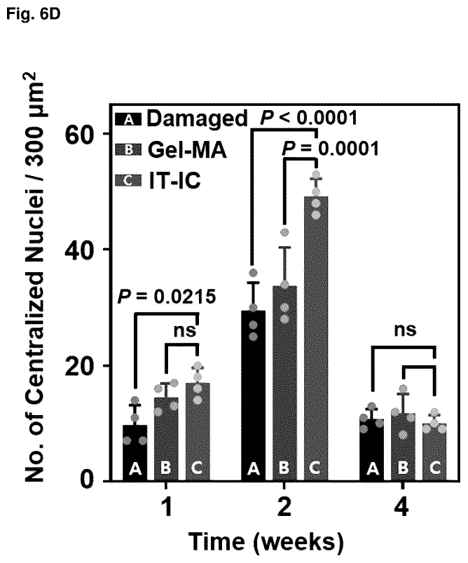

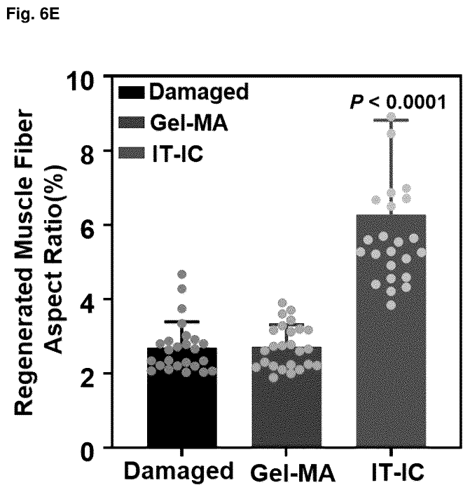

The results are shown in FIG. 6a to 6d.

Filling the cylindrical defect area in the anterior tibialis muscle with IT-IC hydrogel led to successful muscle tissue regeneration within four weeks (FIG. 5a).

Histological analysis using H&E staining revealed a significant increase in the number of myofibers with central nuclei at 1 and 2 weeks in the IT-IC hydrogel group compared to the untreated and Gel-MA groups (FIGS. 6b and 6d). Furthermore, the aspect ratio of regenerated myofibers measured after 4 weeks was significantly improved in the IT-IC hydrogel group compared to other groups (FIGS. 6c and 6e).

Taken together, the data demonstrates that the muscle-tissue interfacing with IT-IC hydrogel enables immediate electrical conduction between tissues in the early stages after injection and facilitates additional tissue recovery (healing/repair) in the later stages.

### Example 2: Construction and Evaluation of Gait Rehabilitation Assistance Device

### Example 2.1: Construction of Gait Rehabilitation Assistance Device

To construct a closed-loop robot-assisted rehabilitation device (C-RAR) using conductive hydrogel (IT-IC), IT-IC hydrogel was injected onto the surface of the anterior tibialis muscle and sciatic nerve in rat with volumetric muscle loss (VML). Subsequently, inherently stretchable electrodes prepared for electrical stimulation delivery were conformally mounted on the TA muscle and sciatic nerve. The stretchable devices, used as cuff-shaped neural interfaces or epimysial electrodes, were connected to external interconnects fixed to the head of the IT-IC hydrogel-treated VML mouse. External interconnects were connected to a waveform generator and data acquisition device. Wire-shaped electrodes were inserted into the rat's tail and connected to the data acquisition device. Based on this setup, the robot was designed to assist the rat's gait while walking on a treadmill.

The sciatic nerve of the rat received electrical stimulation every 3 seconds, delivered as specific pulses (1 ms duration) from the waveform generator. This stimulation was applied to a rat walking on the treadmill at a speed of 3 m/min and was adjusted based on the magnitude of the EMG signals.

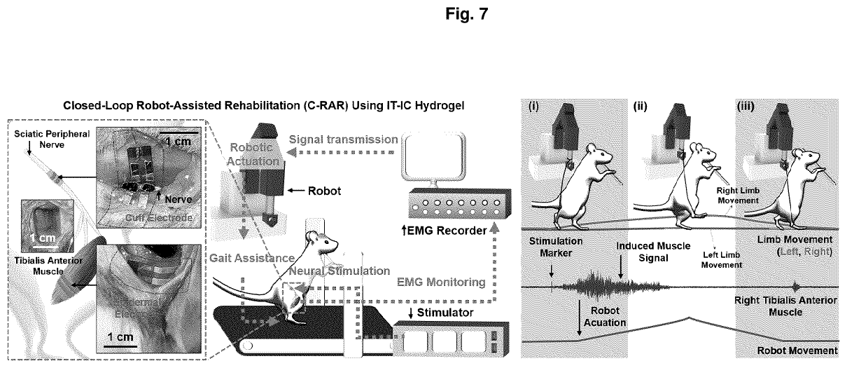

FIG. 7 illustrates a mouse using the closed-loop robot-assisted rehabilitation device (C-RAR) with IT-IC hydrogel in the schematic view.

### Example 2.2: Evaluation of Gait Rehabilitation Assistance Device

This example evaluated the effectiveness of the gait rehabilitation assistance device constructed in Example 2.1 three days post-VML surgery in awake rat.

In brief, initial parameters were set based on electrical simulation (initial stimulation amplitude: 50 mV, update weight: 5, EMG threshold: 15×, and window size: 5k samples every 50 ms). EMG signals were recorded in real-time at a sampling frequency of 100 KHz. These EMG signals were simultaneously processed with a band-pass filter (200-1500 Hz) to remove noise caused by rat movements. The processed EMG signals were matched using LabChart 8 Pro software, and the absolute values of the filtered signals over 50 ms were calculated in real-time. Using the LabChart-MATLAB application programming interface (API) and MATLAB-Dobot Studio API, custom code in MATLAB software calculated the average EMG signal. If the averaged EMG signal fell below a predefined threshold voltage, the amplitude of electrical stimulation was increased in increments of 10 mV. If the averaged EMG signal exceeded the threshold, a cue was activated to initiate robot-assisted support at a z-axis height of 2 cm, and the stimulation amplitude was adjusted proportionally to the change in the averaged EMG signal.

Recorded videos for were manually quantitatively analyzed for gait cycle percentages (stance and swing phases), maximum toe height, and stride length. All video analysis was conducted using Adobe Premiere Pro v.22.5, PotPlayer v. 1.7.21831, and VapMix2.

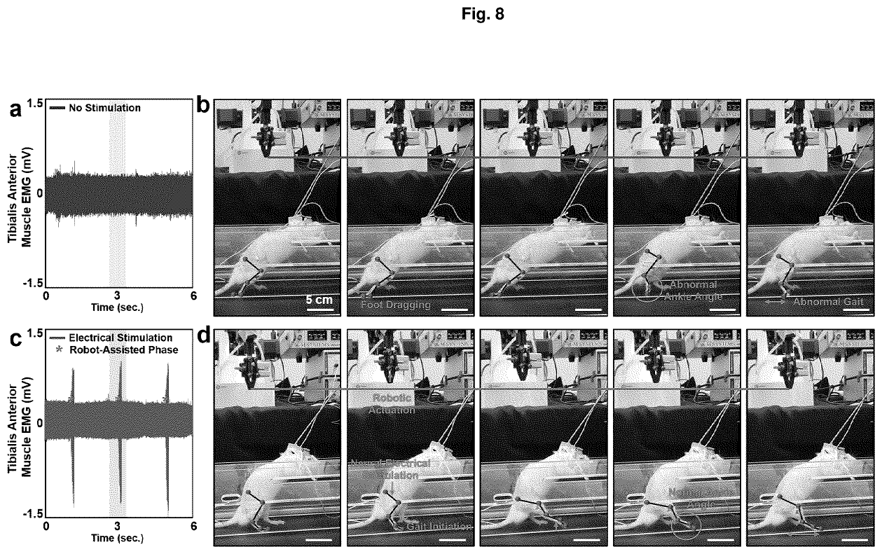

The results are shown in FIG. 8.

First, as shown in FIG. 8 (a) and 8 (b), in the control group (no nerve stimulation), only minimal TA EMG signals were generated due to the lack of spontaneous contraction in the damaged muscle tissue. These signals did not exceed the threshold voltage, leading to gait impairments such as dragging of the foot, irregular toe angles, and poor walking patterns.

When nerve stimulation was applied to the right leg of the mouse, composite EMG signals exceeding the threshold voltage were clearly detected. This triggered external robotic assistance, enabling the mouse to achieve normal walking.

These findings demonstrate that applying IT-IC hydrogel to damaged muscle tissues enables electrical conduction, facilitating the immediate operation of the gait rehabilitation assistance device. This highlights the potential of the C-RAR device as an effective robot-based rehabilitation system.

What is claimed is:

1. A muscle or nerve function rehabilitation assistance device (1), comprising:
(a) a conductive hydrogel to be injected into a muscle injury site or nerve injury site of a subject;
(b) a neural stimulation electrode 100 for delivering electrical stimulation to the nerves of the subject;
(c) an electrical stimulation generator 200 for applying a stimulation signal to the neural stimulation electrode;
(d) an electromyographic electrode 300 for measuring an electromyographic signal of the target rehabilitation muscle of the subject; and
(e) an electromyographic recorder 400 for receiving an electromyographic signal from the electromyographic electrode.

2. The rehabilitation assistance device (1) of claim 1, further comprising: (f) a motor function assistance device (500) for motor function rehabilitation of muscles.

3. The rehabilitation assistance device (1) of claim 2, wherein the motor function assistance device (500) comprises:
an actuator (503) for providing motion assistance force in a flexion-extension direction of the muscle of the subject;
a body fixation unit (504) for securing the subject's body part;
a connecting member (505) for transmitting force by linking the actuator and the body fixation unit; and
a power control unit (506) for determining whether to supply power to the actuator.

4. The rehabilitation assistance device (1) of claim 1, wherein the motor function assistance device (500) further comprises a treadmill (501) for gait training and a weight support (502) for supporting the weight of the subject.

5. The rehabilitation assistance device (1) of claim 1, wherein the conductive hydrogel comprises hyaluronic acid and noble metal nanoparticles,
the hyaluronic acid forming a network linked by biphenyls; and
the noble metal nanoparticles being dispersed within the network.

6. The rehabilitation assistance device (1) of claim 1, wherein the conductive hydrogel has a resistance of 1 kΩ to 100 kΩ.

7. The rehabilitation assistance device (1) of claim 1, wherein the conductive hydrogel has a tangent delta (tan(δ)) of 0 (exclusive) to 0.5 (inclusive).

8. The rehabilitation assistance device (1) of claim 1, wherein the conductive hydrogel has an elongation in longitudinal direction of 80% to 120%.

9. The rehabilitation assistance device (1) of claim 1, wherein the electromyographic recorder (400) monitors and records electromyographic signals (EMG) and transmits the signals to the electrical stimulation generator (200) and the power control unit (506) of the motor function assistance device.

10. The rehabilitation assistance device (1) of claim 9, wherein the electromyographic recorder removes noise signals caused by the subject's movements by filtering the collected EMG signals.

11. The rehabilitation assistance device (1) of claim 9, wherein the electrical stimulation generator (200) controls the intensity of the generated electrical stimulation signals by comparing the target EMG signal value determined through electrical simulation with the measured EMG signal value, such that: i) when the EMG signal is below the target EMG signal value, the magnitude of the electrical stimulation signal applied to the neural stimulation electrode is increased; and ii) when the EMG signal exceeds the target EMG signal value, the magnitude of the electrical stimulation signal applied to the neural stimulation electrode is decreased.

12. The rehabilitation assistance device (1) of claim 9, wherein the power control unit (506) controls the intensity of the provided motion assistance force by comparing the target electromyographic signal value determined through electrical simulation with the measured electromyographic signal value, such that: i) when the electromyographic signal is below the target electromyographic signal value, motion assistance signals are transmitted to the motor function assistance device (500) to increase the magnitude of the motion assistance force; and ii) when the electromyographic signal exceeds the target electromyographic signal value, motion assistance signals are transmitted to the motor function assistance device (500) to decrease the magnitude of the motion assistance force.

13. The rehabilitation assistance device (1) of claim 3, wherein the actuator (503) is a linear actuator operating in the flexion-extension direction of the muscle.

14. The rehabilitation assistance device (1) of claim 3, wherein the actuator (503) provides motion assistance force by operating in response to motion assistance signals received from the power control unit (506).
